(19) United States
(12) Patent Application Publication
(10) Pub. No.: US 2020/0071754 A1
(43) Pub. Date: Mar. 5, 2020

(54) METHODS AND SYSTEMS FOR DETECTING CONTAMINATION BETWEEN SAMPLES

(71) Applicant: GUARDANT HEALTH, INC., Redwood City, CA (US)

(72) Inventors: Darya CHUDOVA, San Jose, CA (US); Helmy ELTOUKHY, Atherton, CA (US); Stephen FAIRCLOUGH, Redwood City, CA (US); Narsi RAJAGOPALAN, Redwood City, CA (US); Marcin SIKORA, Redwood City, CA (US)

(21) Appl. No.: 16/557,931

(22) Filed: Aug. 30, 2019

Related U.S. Application Data
(60) Provisional application No. 62/724,622, filed on Aug. 30, 2018.

(51) Int. Cl. C12Q 1/6869; G16B 30/10; G16B 20/00

(52) U.S. Cl. CPC C12Q 1/6869; G16B 20/00; G16B 30/10

(57) ABSTRACT

Provided herein are various methods and related systems for detecting the presence/absence of contamination of a first sample with a second sample. In some embodiments, for example, the methods include (a) sequencing a set of polynucleotides to produce a plurality of sequencing reads, (b) aligning the plurality of sequencing reads to a reference sequence, (c) grouping the plurality of sequencing reads into a plurality of families, (d) generating family identifiers for the plurality of families, (e) screening for a set of shared family identifiers, (f) determining a quantitative measure of the set of shared family identifiers, and (g) classifying the first sample as being contaminated or not contaminated with the second sample based on the quantitative measure of the shared family identifiers.

### CROSS-REFERENCE

This application claims the benefit of, and priority to, U.S. Provisional Application No. 62/724,622, filed on Aug. 30, 2018, which application is incorporated herein by reference in its entirety.

### BACKGROUND

Cancer is usually caused by the accumulation of mutations within an individual's normal cells, at least some of which result in improperly regulated cell division. Such mutations commonly include single nucleotide variations (SNVs), gene fusions, insertions and deletions (indels), transversions, translocations, and inversions.

Cancers are often detected by tissue biopsies of tumors followed by analysis of cell pathologies, biomarkers or DNA extracted from cells. But recently it has been proposed that cancers can also be detected from cell-free nucleic acids (e.g., circulating nucleic acids, circulating tumor nucleic acids, exosomes, nucleic acids from apoptotic cells and/or necrotic cells) in bodily fluids, such as blood or urine (see, e.g., Siravegna et al., Nature Reviews, 14:531-548 (2017)). Such tests have the advantage that they are non-invasive, can be performed without identifying suspected cancer cells to biopsy and sample nucleic acids from all parts of a cancer. However, such tests are complicated by the fact that the amount of nucleic acids released into bodily fluids is low and variable as is recovery of nucleic acids from such fluids in analyzable form. These tests are designed such that they can detect very low frequency sequences, represented by as few as 1 in 1000 molecules at a given locus. Hence, such tests could be susceptible to false positive results based on low levels of contamination of molecules from other samples.

The samples can be contaminated by a variety of sources, such as, but not limited to: physical carryover of liquids between samples (e.g. pipetting, automated liquid handling via sample prep or sequencer, manipulating amplified material); demultiplexing artifacts (e.g. base call errors confounding sample indexes that have limited pairwise Hamming distance; insertion/deletion confounding sample indexes that have limited pairwise edit distance) and reagent impurities (e.g. sample index oligos that have some level of missing of oligos synthesized in the same batch; sample index oligos contaminated (through either carryover of synthesis errors) with oligos containing another sample index).

### SUMMARY

This application discloses methods and systems for detecting contamination between two samples. Previous methods of contamination detection in samples are based on the detection of certain molecules, which in uncontaminated samples can only be present in high abundance or not at all, but if observed in low abundance are indicative of contamination. Two such types of molecules are molecules carrying common germline single nucleotide polymorphisms (SNPs) or Y chromosome molecules. These methods are limited by the fact that the above molecules are typically only a small fraction of overall contaminating molecules, and their quantity may be insufficient for detection in presence of sequencing errors and sampling errors. Furthermore, at high contamination rates, the contamination-based germline SNVs may be indistinguishable from germline SNVs native to the contaminated sample. Using Y chromosome molecules as a mechanism of detection is further limited to contamination of female patient samples by male patient samples as Y chromosome molecules are naturally present only in male patients. In addition to physical contamination, digital cross-contamination may occur when a sample index is easily transformed into another index that is then mis-assigned algorithmically. This problem can be mitigated by dual indexing, but that method has its own drawbacks.

The present disclosure provides methods, compositions, and systems for detecting the presence or absence of contamination of a first sample with a second sample.

In one aspect, the present disclosure provides a system for detecting contamination the presence or absence of contamination of first sample with second sample, comprising: a communication interface that receives, over a communication network, a plurality of sequencing reads of a set of tagged polynucleotides from the samples generated by a nucleic acid sequencer, wherein the sequencing read comprises a tag sequence and sequence derived from a polynucleotide; and a computer in communication with the communication interface, wherein the computer comprises one or more computer processors and a computer readable medium comprising machine-executable code that, upon execution by the one or more computer processors, implements a method comprising: (a) receiving, over the communication network, the plurality of sequencing reads of the set of tagged polynucleotides from the samples generated by the nucleic acid sequencer; (b) aligning the plurality of sequencing reads to a reference sequence whereby a beginning region and an end region of the alignment is determined; (c) for each sample, grouping the plurality of sequencing reads into a plurality of families based on grouping features, which comprise from at least one of (i) the tag, (ii) the beginning region, (iii) the end region and (iv) length of the polynucleotide, wherein each family in the sample comprises sequencing reads of tagged progeny polynucleotides amplified from a unique polynucleotide among the set of polynucleotides in the sample; (d) generating family identifiers for the plurality of families; (e) screening for a set of shared family identifiers wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold.

In another aspect, the present disclosure provides a system comprising a controller comprising or capable of accessing, computer readable media comprising non-transitory computer-executable instructions which, when executed by at least one electronic processor perform a method comprising: (a) sequencing a set of polynucleotides from a first sample and a second sample to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) generating family identifiers for the plurality of families; (e) screening for a set of shared family identifiers, wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold.

In another aspect, the present disclosure provides a system, comprising a controller comprising or capable of accessing, computer readable media comprising non-transitory computer-executable instructions which, when executed by at least one electronic processor perform a method comprising: (a) sequencing a set of polynucleotides from the samples to produce a plurality of sequencing reads; (b) aligning the plurality of sequencing reads to a reference sequence whereby a beginning region and an end region of the alignment is determined; (c) grouping the plurality of sequencing reads of the two samples together into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) screening for the plurality of families to identify a set of shared families; wherein a given shared family comprises at least one sequencing read from the first sample and at least one sequencing read from the second sample; (e) determining a quantitative measure derived from the set of shared families; (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In another aspect, the present disclosure provides a system, comprising a controller comprising or capable of accessing, computer readable media comprising non-transitory computer-executable instructions which, when executed by at least one electronic processor perform a method comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature, which comprises the tag, wherein each family in the sample comprises sequencing reads of tagged progeny polynucleotides amplified from a unique polynucleotide among the set of tagged polynucleotides in the sample; (e) screening for a set of shared family identifiers wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or as not being contaminated with the second sample if the quantitative measure of shared family identifiers is at or below the predetermined threshold.

In another aspect, the present disclosure provides a system, comprising a controller comprising or capable of accessing, computer readable media comprising non-transitory computer-executable instructions which, when executed by at least one electronic processor perform a method comprising: (a) sequencing a set of polynucleotides from the samples to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) screening for the plurality of families to identify a set of shared families; wherein a given shared family is a family of the first sample with grouping features that are identical or substantially identical to grouping features of a family of the second sample; (e) determining a quantitative measure of the set of shared families for the first sample; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In some embodiments, the sequencing read comprises (i) a tag sequence, and (ii) a sequence derived from the polynucleotide. In some embodiment the system further comprises for each sample, grouping the plurality of sequencing reads into a plurality of families based on information from at least one of (i) the tag, (ii) the beginning region, (iii) the end region and (iv) length of the polynucleotide, wherein each family in the sample comprises sequencing reads of progeny polynucleotides amplified from a unique polynucleotide among the set of polynucleotides in the sample.

In another aspect, the present disclosure provides a system, comprising a controller comprising or capable of accessing, computer readable media comprising non-transitory computer-executable instructions which, when executed by at least one electronic processor perform a method comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature, which comprises the tag, wherein each family in the sample comprises sequencing reads of tagged progeny polynucleotides amplified from a unique polynucleotide among the set of tagged polynucleotides in the sample; (d) screening for the plurality of families to identify a set of shared families; wherein a given shared family is a family of the first sample with grouping features that are identical or substantially identical to grouping features of a family of the second sample; (e) determining a quantitative measure of the set of shared families for the first sample; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In another aspect, the present disclosure provides a system, comprising a controller comprising or capable of accessing, computer readable media comprising non-transitory computer-executable instructions which, when executed by at least one electronic processor perform a method comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) grouping the plurality of sequencing reads of the two samples together into a plurality of families based on grouping feature, which comprises the tag, wherein each family in the sample comprises sequencing reads of tagged progeny polynucleotides amplified from a unique polynucleotide among the set of tagged polynucleotides in the sample; (d) screening for the plurality of families to identify a set of shared families, wherein a given shared family comprises at least one sequencing read from the first sample and at least one sequencing read from the second sample; (e) determining a quantitative measure derived from the set of shared families; (f) classifying the first sample as being contaminated with the second sample if the quantitative measure of the set of shared families is above a predetermined threshold, or as not being contaminated with the second sample if the quantitative measure of shared families is at or below the predetermined threshold.

In some embodiments, the system further comprises detecting a somatic genetic variation of the polynucleotides of the first sample by excluding the sequencing reads of the shared families of the first sample, wherein the first sample is classified as being contaminated with the second sample.

In some embodiments, the system further comprises generating a report which optionally includes information on, and/or information derived from, the contamination status of the sample.

In some embodiments, the system further comprises communicating the report to a third party, such as the subject from whom the sample derived or a health care practitioner

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based grouping features, which comprise at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) generating family identifiers for the plurality of families; (e) screening for a set of shared family identifiers wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or not being contaminated with the second sample if the quantitative measure of the set of shared family identifiers is at or below the predetermined threshold.

In another aspect, the present disclosure provides a method for detecting the presence or absence of contamination of a first sample with a second sample, comprising: (a) accessing, by a computer system, sequence information comprising a plurality of sequencing reads from the first and second sample; (b) aligning, by the computer system, the plurality of sequencing reads to a reference sequence whereby a beginning region and an end region of the alignment is determined; (c) for each sample, grouping, by the computer system, the plurality of sequencing reads into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among a set of polynucleotides in the sample (d) generating, by the computer system, family identifiers for the plurality of families; (e) screening, by the computer system, for a set of shared family identifiers wherein a given shared family identifier is a family identifier of the first sample that is identical or substantially identical to a family identifier of the second sample; (f) determining, by the computer system, a quantitative measure of the set of shared family identifiers; and (g) classifying, by the computer system, the first sample as being contaminated with the second sample if the quantitative measure of the set of shared family identifiers is above a predetermined threshold, or as not being contaminated with the second sample if the quantitative measure of the set of shared family identifiers is at or below the predetermined threshold.

In another aspect, the present disclosure provides a method for detecting the presence or absence of contamination of a first sample with a second sample, comprising: (a) obtaining sequence information comprising a plurality of sequencing reads from the first and second sample; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among a set of polynucleotides in the sample; (d) generating family identifiers for the plurality of families; (e) screening for a set of shared family identifiers, wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold.

In some embodiments, the method further comprises, prior to a), tagging the set of polynucleotides to generate tagged polynucleotides, wherein each tagged polynucleotide comprises a tag and a polynucleotide. In some embodiments, the method further comprises, for each sample, grouping the plurality of sequencing reads into a plurality of families based on grouping features, which comprise at least one of (i) the tag, (ii) the beginning region, (iii) the end region and (iv) length of the polynucleotide, wherein each family in the sample comprises sequencing reads of progeny polynucleotides amplified from a unique polynucleotide among the set of polynucleotides in the sample.

In another aspect, the present disclosure provides a method for detecting the presence or absence of contamination of a first sample with a second sample, comprising: (a) sequencing a set of tagged polynucleotides or polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature, which comprises the tag, wherein each family in the sample comprises sequencing reads of tagged progeny polynucleotides amplified from a unique polynucleotide among the set of tagged polynucleotides in the sample; (d) generating family identifiers for the plurality of families; (e) screening for a set of shared family identifiers, wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the shared family identifiers is above a predetermined threshold, or as not being contaminated with the second sample if the quantitative measure of the set of shared family identifiers is at or below the predetermined threshold.

In some embodiments, wherein the quantitative measure of the set of shared family identifiers is a number of shared family identifiers in the first sample. In some embodiments, the quantitative measure of the set of shared family identifiers comprises a ratio of number of shared family identifiers in the first sample to a total number of family identifiers in the first sample. In some embodiments, the quantitative measure of the set of shared family identifiers excludes those shared family identifiers in the first sample for which the number of sequencing reads in the family of the first sample is greater than the number of sequencing reads in the corresponding family of the second sample. In some embodiments, the quantitative measure of the set of shared family identifiers in the first sample excludes shared family identifiers at over-represented pairs of genomic start positions and genomic stop positions. In some embodiments, the total number of family identifiers in the first sample excludes family identifiers at the over-represented pairs of genomic start positions and genomic stop positions.

In some embodiments, the over-represented pairs of genomic start positions and genomic stop positions are determined by: (a) providing a plurality of samples, wherein the plurality of samples comprises a distribution of genomic start positions and genomic stop positions that are identical or substantially identical to the first sample and/or the second sample; (b) determining family identifiers in the plurality of samples; (c) quantifying number of family identifiers in the plurality of samples sharing a pair of genomic start position and genomic stop position; and (d) categorizing the pair of genomic start position and genomic stop position as over-represented if the number of family identifiers exceeds a set threshold. In some embodiments, the plurality of samples excludes the first sample or the second sample. In some embodiments, the plurality of samples excludes the first sample and the second sample. In some embodiments, the plurality of samples comprises samples processed in the same flow cell as the first sample. In some embodiments, the plurality of samples comprises training samples. In some embodiments, the set threshold is at least 2, at least 3, at least 4, at least 5, at least 6, at least 7, at least 8, at least 10, at least 15, at least 20, at least 25, at least 30, at least 35, at least 40, at least 45, at least 50, at least 55 or at least 60 families.

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) screening for the plurality of families to identify a set of shared families, wherein a given shared family is a family of the first sample with grouping features that are identical or substantially identical to grouping features of a family of the second sample; (e) determining a quantitative measure of the set of shared families for the first sample; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) grouping the plurality of sequencing reads of the two samples together into a plurality of families based on grouping features, which comprise at least one of (i) the beginning region, (ii) the end region and (iii) length of the polynucleotide, wherein each family comprises sequencing reads of progeny polynucleotides amplified from a unique polynucleotide among the set of polynucleotides; (d) screening for the plurality of families to identify a set of shared families; wherein the shared family comprises at least one sequencing read from the first sample and at least one sequencing read from the second sample; (e) determining a quantitative measure derived from the set of shared families; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In some embodiments, the method further comprises, prior to the sequencing, tagging a set of polynucleotides to generate tagged polynucleotides, wherein each tagged polynucleotide comprises a tag and a polynucleotide.

In some embodiment, the method comprises, for each sample, grouping the plurality of sequencing reads into a plurality of families based on grouping features, which comprise at least one of (i) the tag, (ii) the beginning region, (iii) the end region and (iv) length of the polynucleotide, wherein each family in the sample comprises sequencing reads of progeny polynucleotides amplified from a unique polynucleotide among the set of polynucleotides in the sample.

In another aspect, the present disclosure provides a method for detecting the presence or absence of contamination of a first sample with a second sample, comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature, which comprises the tag, wherein the family in the sample comprises sequencing reads of tagged progeny polynucleotides amplified from a unique polynucleotide among the set of tagged polynucleotides in the sample; (d) screening for the plurality of families to identify a set of shared families; wherein a given shared family is a family of the first sample with grouping features that are identical or substantially identical to grouping features of a family of the second sample; (e) determining a quantitative measure of the set of shared families for the first sample; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In another aspect, the present disclosure provides a method for detecting the presence or absence of contamination of a first sample with a second sample, comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) grouping the plurality of sequencing reads of the two samples together into a plurality of families based on grouping feature, which comprises the tag, wherein each family in the sample comprises sequencing reads of tagged progeny polynucleotides amplified from a unique polynucleotide among the set of tagged polynucleotides in the sample; (d) screening for the plurality of families to identify a set of shared families; wherein a given shared family comprises at least one sequencing read from the first sample and at least one sequencing read the second sample; (e) determining a quantitative measure derived from the set of shared families; and (f) classifying the first sample as being contaminated with the second sample if the quantitative measure of the set of shared families is above a predetermined threshold, or as not being contaminated with the second sample if the quantitative measure of the set of shared families is at or below the predetermined threshold.

In some embodiments, the quantitative measure comprises the number of shared families in the first sample. In some embodiments, the quantitative measure comprises a ratio of number of sequencing reads of the first sample to number of sequencing reads of the second sample in the shared family. In some embodiments, the quantitative measure comprises a ratio of number of shared families in the first sample to a total number of families in the first sample. In some embodiments, the quantitative measure of the set of shared families excludes those shared families in the first sample for which the number of sequencing reads in the family of the first sample is greater than the number of sequencing reads in the corresponding family of the second sample. In some embodiments, the quantitative measure of the set of shared families in the first sample excludes shared families at over-represented pairs of genomic start positions and genomic stop positions. In some embodiments, the total number of families in the first sample excludes families at the over-represented pairs of genomic start positions and genomic stop positions. In some embodiments, the over-represented pairs of genomic start positions and genomic stop positions are determined by: (a) providing a plurality of samples, wherein the plurality of samples comprises a distribution of genomic start positions and genomic stop positions that are identical or substantially identical to the first sample and/or the second sample; (b) determining the families in the plurality of samples; (c) quantifying number of families in the plurality of samples sharing a pair of genomic start position and genomic stop position; and (d) categorizing the pair of genomic start position and genomic stop position as over-represented if the number of families exceeds a set threshold. In some embodiments, the plurality of samples excludes the first sample or the second sample. In some embodiments, the plurality of samples excludes the first sample and the second sample. In some embodiments, the plurality of samples comprises samples processed in the same flow cell as the first sample. In some embodiments, the plurality of samples comprises training samples. In some embodiments, the set threshold is at least 2, at least 3, at least 4, at least 5, at least 6, at least 7, at least 8, at least 10, at least 15, at least 20, at least 25, at least 30, at least 35, at least 40, at least 45, at least 50, at least 55 or at least 60 families. In some embodiments, the set threshold is about 5 families. In some embodiments, the set threshold is about 10 families. In some embodiments, the set threshold is about 15 families. In some embodiments, the set threshold is about 20 families. In some embodiments, the set threshold is about 30 families. In some embodiments, the set threshold is about 40 families. In some embodiments, the set threshold is about 50 families. In some embodiments, the set threshold can be at least 10⁻³, at least 10⁻⁴, at least 10⁻⁵, at least 10⁻⁶, at least 10⁻⁷, at least 10⁻⁸, or at least 10⁻⁹ of total families observed in the plurality of samples. In some embodiments, the set threshold can be about 10⁻⁴ of total families observed in the plurality of samples. In some embodiments, the set threshold can be about 10⁻⁵ of total families observed in the plurality of samples. In some embodiments, the set threshold can be about 10⁻⁶ of total families observed in the plurality of samples. In some embodiments, the set threshold can be about 10⁻⁷ of total families observed in the plurality of samples. In some embodiments, the set threshold can be about 10⁻⁸ of total families observed in the plurality of samples.

In some embodiments, the beginning region comprises a genomic start position of the sequencing read at which the 5′ end of the sequencing read is determined to start aligning to reference sequence and the end region comprises a genomic stop position of the sequencing read at which the 3′ end of the sequencing read is determined to stop aligning to the reference sequence. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence.

In some embodiments, the tag comprises one or more molecular barcodes attached to ends of the polynucleotide. In some embodiments, the one or more molecular barcodes is at least 2, at least 4, at least 5, at least 6, at least 8, at least 10, at least 15 or at least 20 nucleotides in length. In some embodiments, the one or more molecular barcodes attached to the polynucleotides of the first sample are different from the one or more molecular barcodes attached to the polynucleotides of the second sample. In some embodiments, the polynucleotides of the sample are tagged with at least 5, at least 10, at least 15, at least 20, at least 50, at least 100, at least 500, at least 1000, at least 5000, at least 10,000, at least 50,000 or at least 100,000 different molecular barcodes.

In some embodiments, the first sample and the second sample are sequenced in the same flow cell. In some embodiments, the second sample is sequenced in a different flow cell than the first sample. In some embodiments, the second sample is processed on the same day as of the first sample, but at a different time than the first sample. In some embodiments, the second sample is processed at least 1 minute, at least 30 minutes, at least 1 hour, at least 2 hours, at least 3 hours or at least 4 hours after the first is processed. In some embodiments, the first sample and the second sample are processed on different days. In some embodiments, the first sample and the second sample are in a same batch of samples. In some embodiments, the second sample is processed with a same batch of reagents as the first sample. In some embodiments, the first sample and the second sample are processed at different geographic locations.

In some embodiments, the set of tagged polynucleotides of the samples are uniquely tagged. In some embodiments, the set of tagged polynucleotides of the samples are non-uniquely tagged. In some embodiments, the first sample is obtained from a bodily fluid of a subject and the second sample is obtained from a bodily fluid of another subject.

In some embodiments, the polynucleotides are cell-free polynucleotides. In some embodiments, the cell-free polynucleotides are cell-free DNA. In some embodiments, at least one of the subjects have a disease. In some embodiments, the disease is cancer.

In some embodiments, the set of polynucleotides of the samples are amplified prior to sequencing, thereby producing amplified progeny polynucleotides. In some embodiments, the method further comprises selectively enriching at least a portion of the amplified progeny polynucleotides for regions from the subject's genome or transcriptome prior to the sequencing. In some embodiments, the method further comprises attaching one or more sample indexes to one or both ends of the amplified progeny polynucleotides prior to sequencing, wherein the sample indexes distinguishes the first sample and the second sample. In some embodiments, the predetermined threshold is at least 0.001%, at least 0.005%, at least 0.01%, at least 0.05%, at least 0.1%, at least 0.5%, at least 1%, at least 2%, at least 5%, or at least 10% of total number of families in the first sample. In some embodiments, the predetermined threshold is about 0.01% of total number of families in the first sample. In some embodiments, the predetermined threshold is about 0.05% of total number of families in the first sample. In some embodiments, the predetermined threshold is about 0.1% of total number of families in the first sample In some embodiments, the predetermined threshold is about 0.5% of total number of families in the first sample. In some embodiments, the predetermined threshold is about 1% of total number of families in the first sample. In some embodiments, the predetermined threshold is about 2% of total number of families in the first sample.

In some embodiments, the method further comprises detecting a somatic genetic variation of the polynucleotides of the first sample by excluding the sequencing reads of the shared family identifiers of the first sample, wherein the first sample is classified as being contaminated with the second sample. In some embodiments, the method further comprises detecting a somatic genetic variation of the polynucleotides of the first sample by excluding the sequencing reads of the shared families of the first sample, wherein the first sample is classified as being contaminated with the second sample.

In some embodiments, the method further comprises generating a report which optionally includes information on, and/or information derived from, the contamination status of the sample. In some embodiments, the method comprises communicating the report to a third party, such as the subject from whom the sample derived or a health care practitioner.

The embodiments as described herein may be used or applied to both the methods and the systems described herein.

In some embodiments, the results of the systems and/or methods disclosed herein are used as an input to generate a report. The report may be in a paper or electronic format. For example, information on, and/or information derived from, the contamination status of the first sample, as determined by the methods or systems disclosed herein, can be displayed in such a report. The methods or systems disclosed herein may further comprise a step of communicating the report to a third party, such as the subject from whom the sample derived or a health care practitioner.

The various steps of the methods disclosed herein, or the steps carried out by the systems disclosed herein, may be carried out at the same time or different times, and/or in the same geographical location or different geographical locations, e.g. countries. The various steps of the methods disclosed herein can be performed by the same person or different people.

In certain aspects, the present disclosure provides non-transitory computer readable media comprising non-transitory computer-executable instructions which, when executed by at least one electronic processor can perform one or more steps or methods described herein.

In another aspect, the present disclosure provides non-transitory computer readable media comprising non-transitory computer-executable instructions which, when executed by at least one electronic processor can perform at least: (a) obtaining a plurality of sequencing reads of the set of tagged polynucleotides from the samples generated by the nucleic acid sequencer; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the tag, (ii) the beginning region, (iii) the end region and (iv) length of the polynucleotide, wherein each family in the sample comprises sequencing reads of tagged progeny polynucleotides amplified from a unique polynucleotide among the set of polynucleotides in the sample; (d) generating family identifiers for the plurality of families; (e) screening for a set of shared family identifiers, wherein a given shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold.

In certain aspects, the methods, systems and/or computer readable media described herein can be used as a quality control metric for the assay performance and/or to assess the quality of the sequencing data obtained in order to ensure reliable detection of somation variation in the samples.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE FIGURES

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown

FIG. 1 is a flow chart representation of a method for detecting the presence or absence of contamination between two samples according to an embodiment of the disclosure.

FIG. 2 is a flow chart representation of a method for detecting the presence or absence of contamination between two samples according to an embodiment of the disclosure.

FIG. 3 is a schematic diagram illustrating the grouping of sequencing reads into families and thereby detecting the presence or absence of contamination between two samples according to an embodiment of the disclosure.

FIG. 4 is a schematic diagram of an exemplary system suitable for use with some embodiments of the disclosure.

### DEFINITION OF TERMS

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to a short nucleic acid (e.g., less than about 500 nucleotides, less than about 100 nucleotides, or less than about 50 nucleotides in length) that is typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some embodiments, an adapter of the same sequence is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other example embodiments, an adapter is a bell-shaped adapter that includes a blunt or tailed end for joining to a nucleic acid molecule to be analyzed. Other examples of adapters include T-tailed and C-tailed adapters.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” or “molecular barcode” in the context of nucleic acids refers to a nucleic acid molecule comprising a sequence that can serve as a molecular identifier. For example, individual “barcode” sequences are typically added to each DNA fragment during next-generation sequencing (NGS) library preparation so that each read can be identified and sorted before the final data analysis.

Cancer Type: As used herein, “cancer type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Nucleic Acids: As used herein, “cellular nucleic acids” means nucleic acids that are disposed within one or more cells from which the nucleic acids have originated, at least at the point a sample is taken or collected from a subject, even if those nucleic acids are subsequently removed (e.g., via cell lysis) as part of a given analytical process.

Contamination of samples: As used herein, the terms “contamination” or “contamination of samples” refer to any chemical or digital contamination of one sample with another sample. Contamination can be due to a variety of sources, such as, but not limited to: physical carryover of liquids between samples (e.g. pipetting, automated liquid handling via sample preparation or sequencer systems, manipulating amplified material); demultiplexing artifacts (e.g. base call errors confounding sample indexes that have limited pairwise Hamming distance; insertion/deletion confounding sample indexes that have limited pairwise edit distance) and reagent impurities (e.g. sample index oligos contaminated (through either carryover of synthesis errors) with oligos containing another sample index).

Deoxyribonucleic Acid or Ribonucleic Acid: As used herein, “deoxyribonucleic acid” or “DNA” refers to a natural or modified nucleotide which has a hydrogen group at the 2′-position of the sugar moiety. DNA typically includes a chain of nucleotides comprising four types of nucleotide bases; adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising four types of nucleotide bases; A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Family: As used herein, the term “family” refers to one or more sequencing reads that are derived from a single polynucleotide molecule. Bioinformatically, the one or more sequencing reads derived from a single polynucleotide molecule will have identical or substantially identical grouping features, wherein the grouping features comprise at least one of the following: (i) tag (i.e., molecular barcode), (ii) beginning region of the alignment, (iii) end region of the alignment and (iv) length of the polynucleotide. Those sequencing reads that have identical or substantially identical grouping features can be grouped together into a family. In some embodiments, though there is a low probability, at least two molecules can have the same grouping features and hence the sequencing reads derived from the at least two molecules can be grouped into a single family.

In some embodiments, the sequencing reads derived from a single polynucleotide molecule are detected in only a single sample. In some embodiments, where there is contamination of at least two samples, then the sequencing reads derived from a single polynucleotide molecule (of a single sample) can be detected in the at least two samples. In these embodiments, where the grouping of sequencing reads is performed independently for each sample, then the sequencing reads derived from a single polynucleotide molecule that is detected within each sample will be grouped as a separate family in that sample. In other embodiments, where the grouping of sequencing reads is performed together for all the at least two samples, then the sequencing reads derived from a single polynucleotide molecule that are detected in the at least two samples will be grouped into a single family.

The grouping features of the family are representative of the grouping features of the sequencing reads in the family. In some embodiments, if a family comprises sequencing reads with identical grouping features, then the grouping feature of any of the sequencing reads is the grouping feature of the family. In other embodiments, if a family comprises sequencing reads with identical and substantially identical grouping features, the grouping feature of the family can be one or a combination of the following, but not limited to: (i) most frequently represented grouping feature of sequencing reads; (ii) average of the grouping features of the sequencing reads; (iii) most frequently represented nucleotide base in a molecular barcode; (iv) maximum likelihood value of the molecular barcode and/or beginning region and/or end region of the sequencing read.

In some embodiments, the family comprises at least two sequencing reads derived from a single polynucleotide molecule. In some embodiments, the family can comprise sequence reads derived from a single strand of a double-stranded polynucleotide molecule. In some embodiments, the family comprises sequence reads derived from both strands (sense and anti-sense strands) of a double-stranded polynucleotide molecule. In an instance, the molecular barcode, genomic start position and genomic stop position are considered as grouping features of the family. In this instance, if a family has 10 sequence reads and all the sequence reads have identical molecular barcode and genomic start position but the genomic stop position is not identical, then that molecular barcode and genomic start position becomes the grouping feature of the family and for the genomic stop position—the genomic stop position represented by a majority of the sequencing reads in that family will be considered as the genomic stop position of the family (which is part of the grouping feature of the family).

Family identifier: As used herein, the term “family identifier” refers to an identifier that uniquely identifies each family and it comprises the grouping features and/or information derived from the grouping features of the family. In some embodiments, the family identifier can comprise integers, alphabets or a combination of both. In some embodiments, the family identifier is assigned to the sequencing reads in a family.

Germline Mutation: As used herein, the terms “germline mutation” or “germline variation” are used interchangeably and refer to an inherited mutation (i.e., not one arising post-conception). Germline mutations may be the only mutations that can be passed on to the offspring and may be present in every somatic cell and germline cell in the offspring.

Indel: As used herein, “indel” refers to a mutation that involves the insertion or deletion of nucleotides in the genome of a subject.

Mutant Allele Fraction: As used herein, “mutant allele fraction”, “mutation dose,” or “MAF” refers to the fraction of nucleic acid molecules harboring an allelic alteration or mutation at a given genomic position/locus in a given sample. MAF is generally expressed as a fraction or a percentage. For example, an MAF of a somatic variant may be less than 0.15.

Mutation: As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

Neoplasm: As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular barcodes (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag the nucleic acid molecules such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Over-represented pairs of genomic start positions and genomic stop positions: As used herein, the terms “over-represented pairs of genomic start positions and genomic stop positions” or “over-represented pairs” refer to pairs of genomic start positions and genomic stop positions at which the number or frequency of families in a plurality of samples sharing the pair of genomic start position and genomic stop position exceeds a set threshold. In some embodiments, the plurality of samples comprises samples run in the flow cell in which the first sample and the second sample were run. For example, the plurality of samples can be training samples or samples processed in a particular flow cell of the nucleic acid sequencer related to the first sample and/or the second sample being analyzed. In some embodiments, the plurality of samples excludes a first sample and/or a second sample. In some embodiments, the set threshold can be any value between 2 and 100. In some embodiments, the set threshold can be 2, 3, 4, 5, 6, 7, 8, 9, 10, 11, 12, 13, 14, 15, 16, 17, 18, 19, 20, at least 21, at least 25, at least 30, at least 35, at least 40 or at least 50. In some embodiments, the set threshold can be 5. In some embodiments, the set threshold can be 10. In some embodiments, the set threshold can be 15. In some embodiments, the set threshold can be 20. In some embodiments, the set threshold can be at least 10⁻³, at least 10⁻⁴, at least 10⁻⁵, at least 10⁻⁶, at least 10⁻⁷, at least 10⁻⁸, or at least 10⁻⁹ of total families observed in the plurality of samples. In some embodiments, the set threshold can be 10⁻⁴ of total families observed in the plurality of samples. In some embodiments, the set threshold can be 10⁻⁵ of total families observed in the plurality of samples. In some embodiments, the set threshold can be 10⁻⁶ of total families observed in the plurality of samples. In some embodiments, the set threshold can be 10⁻⁷ of total families observed in the plurality of samples. In some embodiments, the set threshold can be 10⁻⁸ of total families observed in the plurality of samples.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g., 3-4, to hundreds of monomeric units. Whenever a polynucleotide is represented by a sequence of letters, such as “ATGCCTG”, it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Reference Sequence: As used herein, “reference sequence” refers to a known sequence used for purposes of comparison with experimentally determined sequences. For example, a known sequence can be an entire genome, a chromosome, or any segment thereof. A reference typically includes at least about 20, at least about 50, at least about 100, at least about 200, at least about 250, at least about 300, at least about 350, at least about 400, at least about 450, at least about 500, at least about 1000, or more than 1000 nucleotides. A reference sequence can align with a single contiguous sequence of a genome or chromosome or can include non-contiguous segments that align with different regions of a genome or chromosome. Examples of reference sequences include, for example, human genomes, such as, hG19 and hG38.

Sample: As used herein, “sample” means anything capable of being analyzed by the methods and/or systems disclosed herein.

Sequencing: As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and identity of monomer units (e.g., nucleotides, etc.) in that polymer.

Shared family: If the grouping of sequencing reads into families is performed independently for first sample and second sample, then the term “shared family” refers to a family in the first sample whose grouping features is identical or substantially identical to the grouping features of a family in the second sample. Alternatively, if the grouping of sequencing reads into families is performed together for both the first sample and second sample, then the term “shared family” refers to a family that comprises at least one sequencing read from the first sample and at least one sequencing read from the second sample.

In some embodiments, where there is contamination of at least two samples, then the sequencing reads derived from a single polynucleotide molecule (of a single sample) can be detected in the at least two samples. In these embodiments, where the grouping of sequencing reads is performed independently for each sample, then the sequencing reads derived from a single polynucleotide molecule that is detected within each sample will be grouped as a separate family in that sample. In these embodiments, the shared family refers to a family in the first sample whose grouping features is identical or substantially identical to the grouping features of a family in the second sample.

Alternatively, in other embodiments, where the grouping of sequencing reads is performed together for all the at least two samples, then the sequencing reads derived from a single polynucleotide molecule that are detected in the at least two samples will be grouped into a single family. In these embodiments, the shared family refers to a family that has at least one sequencing read from the at least two samples.

In some embodiments, the first sample and the second sample can be in the same flow cell or different flow cells.

Shared family identifier: As used herein, the term “shared family identifier” refers to the family identifier of a family in the first sample that is identical or substantially identical to a family identifier of a family in the second sample—i.e., the grouping feature of family in the first sample is identical or substantially identical to the grouping feature of a family in the second sample. In some embodiments, the first sample and the second sample can be in the same flow cell or in different flow cells.

Single Nucleotide Polymorphism: As used herein, the terms “single nucleotide polymorphism” or “SNP” are used interchangeably. They refer to a variation in a single nucleotide that occurs at a specific position in the genome, where each variation is present to some appreciable degree within a population (e.g., greater than about 1%).

Single Nucleotide Variant: As used herein, “single nucleotide variant” or “SNV” means a mutation or variation in a single nucleotide that occurs at a specific position in the genome.

Somatic Mutation: As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Substantially identical: As used herein, the term “substantially identical” refers to two different entities that are 99.9% identical, at least 95% identical, at least 90% identical, at least 85% identical, at least 80% identical, at least 75% identical, at least 70% identical, at least 60% identical or at least 50% identical. For example, when a family in the first sample is substantially identical to a family in the second sample, then the grouping features of the family in the first sample is 99.9% identical, at least 95% identical, at least 90% identical, at least 85% identical, at least 80% identical, at least 75% identical, at least 70% identical, at least 60% identical or at least 50% identical to the grouping features of the family in the second sample. In cases where the entity is the molecular barcode, then the term “substantically identical” refers to two different molecular barcodes that have a Hamming distance or edit distance of less than 1, less than 2, less than 3, less than 4, less than 5, less than 6, less than 7 or less than 8. In cases where the entity is the beginning region or end region, then the term “substantially identical” refers to two different regions that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp or within 25 bp. In cases where the entity is the length of the polynucleotide, then the term “substantially identical” refers to two different lengths that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp, within 25 bp, within 30 bp, within 40 bp or within 50 bp.

Threshold: As used herein, “threshold” refers to a predetermined value used to characterize experimentally determined values of the same parameter for different samples depending on their relation to the threshold. For example, the threshold for the p-value can refer to any predetermined value between 0 and 1 and is used to identify the origin of a nucleic acid variant.

Training samples: As used herein, “training samples” refers to a set of samples with properties, parameters and/or composition similar to the first sample and/or the second sample that is analyzed for the presence or absence of contamination.

Variant: As used herein, a “variant” can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants, however, are acquired variants and usually have a frequency of less than about 0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

### DETAILED DESCRIPTION

### I. General Overview

In processing the samples for analysis, it is possible to introduce a false positive result by the chemical or digital cross-contamination of samples being processed in the same batch or in close temporally and special proximity through the dissemination of a molecule present in a sample to another sample. In cases where cell-free nucleic acids from samples containing contamination or a second genome (ie., other than the subject's genome and arising from, for example, a transplant, a blood transfusion, or a fetus) are assayed, the samples may need additional manual review or even additional sequencing runs to be performed.

The present disclosure provides methods and systems for detecting the presence or absence of contamination in a first sample with a second sample.

In an aspect, the present disclosure provides a method for detecting the presence or absence of contamination of a first sample with a second sample, comprising: (a) accessing, by a computer system, sequence information comprising a plurality of sequencing reads from the first and second sample; (b) aligning, by the computer system, the plurality of sequencing reads to a reference sequence whereby a beginning region and an end region of the alignment is determined; (c) for each sample, grouping, by the computer system, the plurality of sequencing reads into a plurality of families based on grouping features, which comprises at least one of (i) the beginning region, (ii) the end region and (iii) length of the sequence read, wherein each family in the sample comprises sequencing reads of progeny polynucleotides amplified from a unique polynucleotide among a set of polynucleotides in the sample; (d) generating, by the computer system, family identifiers for the plurality of families; (e) screening, by the computer system, for a set of shared family identifiers wherein the shared family identifier is a family identifier of the first sample that is identical or substantially identical to a family identifier of the second sample; (f) determining, by the computer system, a quantitative measure of the set of shared family identifiers; and (g) classifying, by the computer system, the first sample as being contaminated with the second sample if the quantitative measure of the set of shared family identifiers is above a predetermined threshold, or not contaminated if the quantitative measure of shared family identifiers is at or below the predetermined threshold.

In another aspect, the present disclosure provides a method for detecting the presence or absence of contamination of a first sample with a second sample, comprising: (a) obtaining sequence information comprising a plurality of sequencing reads from the first and second sample; (b) aligning the plurality of sequencing reads to a reference sequence whereby a beginning region and an end region of the alignment is determined; (c) for each sample, grouping the plurality of sequencing reads into a plurality of families based on grouping features, which comprises at least one of (i) the beginning region, (ii) the end region and (iii) length of the sequence read, wherein each family in the sample comprises sequencing reads of progeny polynucleotides amplified from a unique polynucleotide among a set of polynucleotides in the sample; (d) generating family identifiers for the plurality of families; (e) screening for a set of shared family identifiers wherein the shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the set of shared family identifiers is above a predetermined threshold, or not contaminated if the quantitative measure of shared family identifiers is at or below the predetermined threshold.

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based on grouping features, which comprises at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (iv) generating family identifiers for the plurality of families; (v) screening for a set of shared family identifiers wherein the shared family identifier is a family identifier of the first sample that is identical or substantially identical to a family identifier of the second sample; (vi) determining a quantitative measure of the set of shared family identifiers; and (vii) classifying the first sample as being contaminated with the second sample if the quantitative measure of the set of shared family identifiers is above a predetermined threshold, or not contaminated if the quantitative measure of shared family identifiers is at or below the predetermined threshold.

In some embodiments, prior to the sequencing or prior to accessing/obtaining the sequence information, the set of polynucleotides are tagged to generate tagged polynucleotides, wherein each tagged polynucleotide comprises a tag and a polynucleotide. In these embodiments, for each sample, the plurality of sequencing reads are grouped into a plurality of families based on grouping features, which comprises at least one of (i) the tag, (ii) the beginning region, (iii) the end region and (iv) length of the polynucleotide, wherein each family in the sample comprises sequencing reads of progeny polynucleotides amplified from a unique polynucleotide among the set of polynucleotides in the sample.

In another aspect, the present disclosure provides a method for detecting the presence or absence of contamination of a first sample with a second sample, comprising: (a) sequencing a set of tagged polynucleotides or polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature, which comprises the tag, wherein each family in the sample comprises sequencing reads of tagged progeny polynucleotides amplified from a unique polynucleotide among the set of tagged polynucleotides in the sample; (d) generating family identifiers for the plurality of families; (e) screening for a set of shared family identifiers wherein the shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the shared family identifiers is above a predetermined threshold, or not contaminated if the quantitative measure of shared family identifiers is at or below the predetermined threshold.

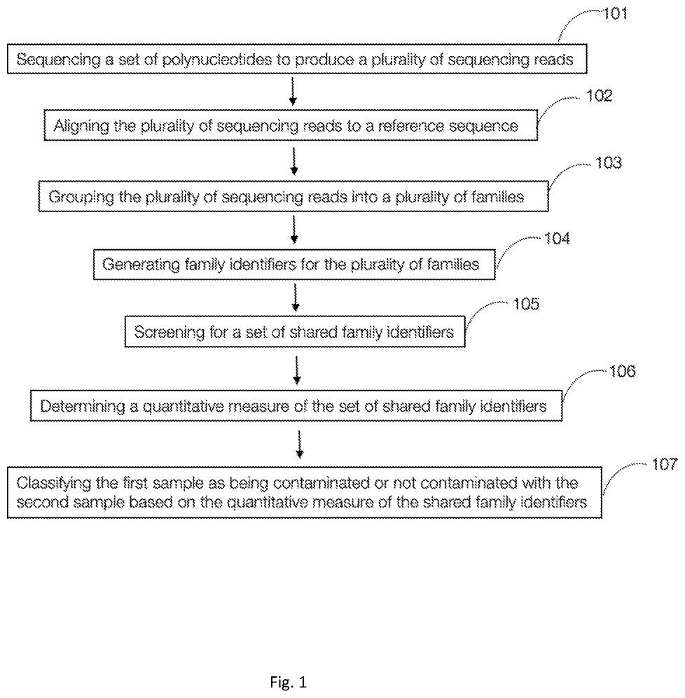

FIG. 1 is a flow chart representation of a method for detecting the presence or absence of contamination between two samples obtained from two different subjects according to an embodiment of the disclosure. The grouping features of the sequencing reads, thereby the grouping features of the family, are used to determined the presence or absence of contamination between two samples. The grouping features of the sequencing reads typically comprise at least one of the following: (i) the tag, (ii) the beginning region, (iii) the end region and (iv) the length of the polynucleotide. In 101, the set of polynucleotides from the samples (i.e., a first sample and a second sample) are sequenced to produce a plurality of sequencing reads. In some embodiments, the first sample and the second sample are sequenced in the same flow cell. In some embodiments, the second sample is sequenced in a different flow cell than the first sample. In some embodiments, the first sample is processed at a different time than the second sample. For example, the second sample is processed at least 1 minute, at least 30 minutes, at least 1 hour, at least 2 hours, at least 3 hours or at least 4 hours after the first is processed. In some embodiments, the first sample and the second sample are processed on different days. In some embodiments, the first sample and the second sample are in a same batch of samples. In some embodiments, the second sample is processed with a same batch of reagents as the first sample. In some embodiments, the first sample and the second sample are processed by the same liquid handling robot. In some embodiments, the first sample and the second sample are processed by the same lab personnel.

In some embodiments, the first sample and the second sample are processed at different geographic locations. In some embodiments, the first sample is obtained from a bodily fluid of a subject and the second sample is obtained from a bodily fluid of another subject. In some embodiments, the sample is blood. In some embodiments, the sample is plasma. In some embodiments, the sample is serum. In some embodiments, the polynucleotides are cell-free polynucleotides. In some embodiments, the cell-free polynucleotides are cell-free DNA. In some embodiments, at least one of the subjects have a disease, such as cancer.

In some embodiments, the set of polynucleotides undergo a series of library preparation steps prior to sequencing. The library preparation steps comprise end repair, ligation of adapters (comprising tags—i.e., molecular barcodes), amplication of tagged polynucleotides and/or selective enrichment of at least a portion of the amplified progeny polynucleotides for regions from the subject's genome or transcriptome. In some embodiments, the first sample and second sample are tagged with tags comprising molecular barcodes to generate a set of tagged polynucleotides. In some embodiments, the set of tagged polynucleotides of the samples are uniquely tagged. In some embodiments, the set of tagged polynucleotides of the samples are non-uniquely tagged. In some embodiments, the method further comprises attaching one or more sample indexes to one or both ends of the amplified progeny polynucleotides prior to sequencing, wherein the sample indexes distinguishes the first sample and the second sample.

For determining the beginning region, end region and/or the length of the polynucleotide, in 102, the plurality of sequencing reads are generally aligned to a reference sequence. The reference sequence can be a human genome. In 103, the plurality of sequencing reads in each sample are grouped into into a plurality of families based on grouping features, which comprise at least one of (i) the tag (if the polynucleotides are tagged), (ii) the beginning region, (iii) the end region and (iv) length of the polynucleotide, wherein each family in the sample comprises sequencing reads of progeny polynucleotides or tagged progeny polynucleotides (in cases where the polynucleotides are tagged with molecular barcodes) amplified from a unique polynucleotide among the set of polynucleotides in the sample. In some embodiments, the beginning region comprises a genomic start position of the sequencing read at which the 5′ end of the sequencing read is determined to start aligning to the reference sequence and the end region comprises a genomic stop position of the sequencing read at which the 3′ end of the sequencing read is determined to stop aligning to the reference sequence. In some embodiments, the beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. In some embodiments, the tag comprises one or more molecular barcodes attached to both ends of a polynucleotide molecule. In some embodiments, the one or more molecular barcodes is at least 2, at least 4, at least 5, at least 6, at least 8, at least 10, at least 15 or at least 20 nucleotides in length. In some embodiments, the polynucleotides of the sample are tagged with at least 5, at least 10, at least 15, at least 20, at least 50, at least 100, at least 500, at least 1000, at least 5000, at least 10,000, at least 50,000 or at least 100,000 different tags/molecular barcodes.

In 104, family identifiers are generated for the plurality of families based on the grouping features. In 105, the family identifiers are screened for a set of shared family identifiers, wherein the shared family identifier is a family identifier of a family in the first sample that is identical or substantially identical to a family identifier of a family in the second sample—i.e., the grouping feature of family in the first sample is identical or substantially identical to the grouping feature of family in the second sample.

In 106, quantitative measure of the set of shared family identifiers is determined in order to classify the sample as being contaminated with another sample or not. In some embodiments, the quantitative measure of the set of shared family identifiers is a number of shared family identifiers in the first sample. In some embodiments, the quantitative measure of the set of shared family identifiers comprises a ratio of number of shared family identifiers in the first sample to total number of family identifiers in the first sample. In some embodiments, the quantitative measure of the set of shared family identifiers excludes those shared family identifiers in the first sample for which the number of sequencing reads in the family of first sample is greater than the number of sequencing reads in the corresponding family of second sample. In some embodiments, the quantitative measure of the set of shared family identifiers in the first sample excludes shared family identifiers at over-represented pairs of genomic start positions and genomic stop positions. In some embodiments, a total number of family identifiers in the first sample excludes the family identifiers at over-represented pairs of genomic start positions and genomic stop positions. In some embodiments, the over-represented pairs of genomic start positions and genomic stop positions are determined by: (a) providing a plurality of samples, wherein the plurality of samples comprises a distribution of genomic start positions and genomic stop positions that are identical or substantially identical to the first sample and/or the second sample; (b) determining family identifiers in the plurality of samples; (c) quantifying number of family identifiers in the plurality of samples sharing a pair of genomic start position and genomic stop position; and (d) categorizing the pair of genomic start position and genomic stop position as over-represented if the number of family identifiers exceeds a set threshold. In some embodiments, wherein the plurality of samples excludes the first sample or the second sample. In some embodiments, the plurality of samples excludes the first sample and the second sample. In some embodiments, the plurality of samples comprises samples processed in the same flow cell as the first sample. In some embodiments, the plurality of samples comprises training samples. In some embodiments, the set threshold is at least 2, at least 3, at least 4, at least 5, at least 6, at least 7, at least 8, at least 10, at least 15, at least 20, at least 25, at least 30, at least 35, at least 40, at least 45, at least 50, at least 55 or at least 60 families. In some embodiments, the set threshold is about 5 families. In some embodiments, the set threshold is about 10 families. In some embodiments, the set threshold is about 15 families. In some embodiments, the set threshold is about 20 families. In some embodiments, the set threshold is about 30 families. In some embodiments, the set threshold is about 40 families. In some embodiments, the set threshold is about 50 families. In some embodiments, the set threshold can be at least 10⁻³, at least 10⁻⁴, at least 10⁻⁵, at least 10⁻⁶, at least 10⁻⁷, at least 10⁻⁸, or at least 10⁻⁹ of total families observed in the plurality of samples. In some embodiments, the set threshold can be about 10⁻⁴ of total families observed in the plurality of samples. In some embodiments, the set threshold can be about 10⁻⁵ of total families observed in the plurality of samples. In some embodiments, the set threshold can be about 10⁻⁶ of total families observed in the plurality of samples. In some embodiments, the set threshold can be about 10⁻⁷ of total families observed in the plurality of samples. In some embodiments, the set threshold can be about 10⁻⁸ of total families observed in the plurality of samples.

In 107, the first sample is classified as being contaminated with the second sample, if the quantitative measure of the shared family identifiers is above a predetermined threshold or not contaminated if the quantitative measure of shared family identifiers is at or below the predetermined threshold. In some embodiments, the predetermined threshold is at least 0.001%, at least 0.005%, at least 0.01%, at least 0.05%, at least 0.1%, at least 0.5%, at least 1%, at least 2%, at least 5%, or at least 10% of total number of families in the first sample. In some embodiments, the predetermined threshold is about 0.01% of total number of families in the first sample. In some embodiments, the predetermined threshold is about 0.05% of total number of families in the first sample. In some embodiments, the predetermined threshold is about 0.1% of total number of families in the first sample. In some embodiments, the predetermined threshold is about 0.5% of total number of families in the first sample. In some embodiments, the predetermined threshold is about 1% of total number of families in the first sample. In some embodiments, the predetermined threshold is about 2% of total number of families in the first sample.

In some embodiments, even if the first sample is classified as being contaminated with the second sample, the method can further allow reliable detection of at least one somatic variation of the polynucleotides of the first sample by excluding the sequencing reads of the shared family identifiers of the first sample prior to the detection of somatic variation.

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) for each sample, grouping the plurality of sequencing reads into a plurality of families based on information from at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) screening for the plurality of families to identify a set of shared families; wherein the shared family is a family of the first sample that is identical or substantially identical to a family of the second sample; (e) determining a quantitative measure of the set of shared families for the first sample; and (f) classifying the first sample as being contaminated with the second sample if the quantitative measure of the shared families is above a predetermined threshold, or not contaminated if the quantitative measure of shared families is at or below the predetermined threshold.

In another aspect, the present disclosure provides a method for detecting the presence or absence of contamination of a first sample with a second sample, comprising: (a) sequencing a set of polynucleotides from the samples to produce a plurality of sequencing reads; (b) aligning the plurality of sequencing reads to a reference sequence whereby a beginning region and an end region of the alignment is determined; (c) grouping the plurality of sequencing reads of the two samples into a plurality of families based on grouping features, which comprises at least one of (i) the beginning region, (ii) the end region and (iii) length of the polynucleotide, wherein each family in the sample comprises sequencing reads of progeny polynucleotides amplified from a unique polynucleotide among the set of polynucleotides in the sample; (d) screening for the plurality of families to identify a set of shared families; wherein the shared family comprises sequencing reads from the first sample and the second sample; (e) determining a quantitative measure derived from the set of shared families; and classifying the first sample as being contaminated with the second sample if the quantitative measure of the shared families is above a predetermined threshold, or not contaminated if the quantitative measure of shared families is at or below the predetermined threshold.

In some embodiments, prior to the sequencing, the set of polynucleotides can be tagged to generate tagged polynucleotides, wherein each tagged polynucleotide comprises a tag and a polynucleotide. In these embodiments, for each sample, the plurality of sequencing reads are grouped into a plurality of families based on grouping features, which comprises at least one of (i) the tag, (ii) the beginning region, (iii) the end region and (iv) length of the polynucleotide, wherein each family in the sample comprises sequencing reads of progeny polynucleotides amplified from a unique polynucleotide among the set of polynucleotides in the sample.

In another aspect, the present disclosure provides a method for detecting the presence or absence of contamination of a first sample with a second sample, comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) for each sample, grouping the plurality of sequencing reads into a plurality of families based on grouping feature that comprises the tag, wherein each family in the sample comprises sequencing reads of tagged progeny polynucleotides amplified from a unique polynucleotide among the set of tagged polynucleotides in the sample; (d) screening for the plurality of families to identify a set of shared families; wherein the shared family is a family of the first sample that is identical or substantially identical to a family of the second sample; (e) determining a quantitative measure of the set of shared families for the first sample; and (f) classifying the first sample as being contaminated with the second sample if the quantitative measure of the shared families is above a predetermined threshold, or not contaminated if the quantitative measure of shared families is at or below the predetermined threshold.

In another aspect, the present disclosure provides a method for detecting the presence or absence of contamination of a first sample with a second sample, comprising: (a) sequencing a set of tagged polynucleotides from the samples to produce a plurality of sequencing reads, wherein each tagged polynucleotide comprises a tag and a polynucleotide; (b) aligning the plurality of sequencing reads to a reference sequence whereby a beginning region and an end region of the alignment is determined; (c) grouping the plurality of sequencing reads of the two samples into a plurality of families based on information from the tag, wherein each family in the sample comprises sequencing reads of tagged progeny polynucleotides amplified from a unique polynucleotide among the set of tagged polynucleotides in the sample; (d) screening for the plurality of families to identify a set of shared families; wherein the shared family comprises sequencing reads from the first sample and the second sample; (e) determining a quantitative measure derived from the set of shared families; and (f) classifying the first sample as being contaminated with the second sample if the quantitative measure of the shared families is above a predetermined threshold, or not contaminated if the quantitative measure of shared families is at or below the predetermined threshold.

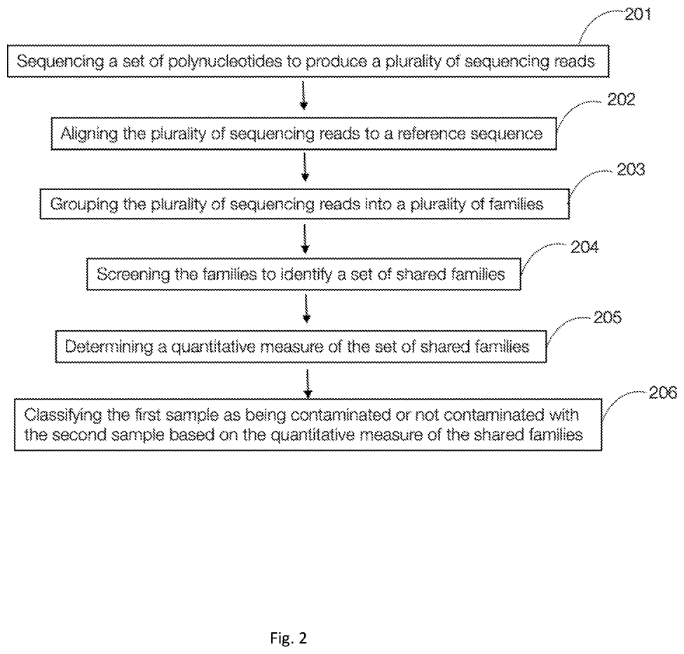

FIG. 2 is a flow chart representation of a method for detecting the presence or absence of contamination between two samples obtained from two different subjects according to an embodiment of the disclosure. The grouping features of the sequencing reads, thereby the grouping features of the family, are used to determined the presence or absence of contamination between two samples. The grouping features of the sequencing reads typically comprise at least one of the following: (i) the tag, (ii) the beginning region, (iii) the end region and (iv) the length of the polynucleotide. In 201, the set of polynucleotides from the samples (i.e., a first sample and a second sample) are sequenced to produce a plurality of sequencing reads. In some embodiments, the first sample and the second sample are sequenced in the same flow cell. In some embodiments, the second sample is sequenced in a different flow cell than the first sample. In some embodiments, the first sample is processed at a different time than the second sample. For example, the second sample is processed at least 1 minute, at least 30 minutes, at least 1 hour, at least 2 hours, at least 3 hours or at least 4 hours after the first is processed. In some embodiments, the first sample and the second sample are processed on different days. In some embodiments, the first sample and the second sample are in a same batch of samples. In some embodiments, the second sample is processed with a same batch of reagents as the first sample.

In some embodiments, the first sample and the second sample are processed at different geographic locations. In some embodiments, the first sample is obtained from a bodily fluid of a subject and the second sample is obtained from a bodily fluid of another subject. In some embodiments, the sample is blood. In some embodiments, the sample is plasma. In some embodiments, the sample is serum. In some embodiments, the polynucleotides are cell-free polynucleotides. In some embodiments, the cell-free polynucleotides are cell-free DNA. In some embodiments, at least one of the subjects have a disease, such as cancer.

In some embodiments, the set of polynucleotides undergo a series of library preparation steps prior to sequencing. The library preparation steps comprise end repair, ligation of adapters (comprising tags—i.e., molecular barcodes), amplication of tagged polynucleotides and/or selective enrichment of at least a portion of the amplified progeny polynucleotides for regions from the subject's genome or transcriptome. In some embodiments, the first sample and second sample are tagged with tags comprising molecular barcodes to generate a set of tagged polynucleotides. In some embodiments, the set of tagged polynucleotides of the samples are uniquely tagged. In some embodiments, the set of tagged polynucleotides of the samples are non-uniquely tagged. In some embodiments, the method further comprises attaching one or more sample indexes to one or both ends of the amplified progeny polynucleotides prior to sequencing, wherein the sample indexes distinguishes the first sample and the second sample.

For determining the beginning region, end region and/or the length of the polynucleotide, in 202, the plurality of sequencing reads are aligned to a reference sequence. The reference sequence can be a human genome (e.g., hg18, hg19). In 203, the plurality of sequencing reads in each sample are grouped into into a plurality of families based on grouping features, which comprise at least one of (i) the tag (if the polynucleotides are tagged), (ii) the beginning region, (iii) the end region and (iv) length of the polynucleotide, wherein each family in the sample comprises sequencing reads of progeny polynucleotides or tagged progeny polynucleotides (in cases where the polynucleotides are tagged with molecular barcodes) amplified from a unique polynucleotide among the set of polynucleotides in the sample. In some embodiments, the beginning region comprises a genomic start position of the sequencing read at which the 5′ end of the sequencing read is determined to start aligning to the reference sequence and the end region comprises a genomic stop position of the sequencing read at which the 3′ end of the sequencing read is determined to stop aligning to the reference sequence. In some embodiments, the beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. In some embodiments, the tag comprises one or more molecular barcodes attached to both ends of a polynucleotide molecule. In some embodiments, the one or more molecular barcodes is at least 2, at least 4, at least 5, at least 6, at least 8, at least 10, at least 15 or at least 20 nucleotides in length. In some embodiments, the polynucleotides of the sample are tagged with at least 5, at least 10, at least 15, at least 20, at least 50, at least 100, at least 500, at least 1000, at least 5000, at least 10,000, at least 50,000 or at least 100,000 different tags/molecular barcodes.

In 204, the plurality of families are screened based on the grouping features for the set of shared families, wherein the shared family is a family in the first sample that is identical or substantially identical to a family in the second sample—i.e., the grouping feature of family in the first sample is identical or substantially identical to the grouping feature of family in the second sample.

In 205, quantitative measure of the set of shared families is determined in order to classify the sample as being contaminated with another sample or not. In some embodiments, the quantitative measure of the set of shared families is a number of shared families in the first sample. In some embodiments, the quantitative measure of the set of shared families comprises a ratio of number of shared families in the first sample to total number of families in the first sample. In some embodiments, the quantitative measure of the set of shared families excludes those shared families in the first sample for which the number of sequencing reads in the family of first sample is greater than the number of sequencing reads in the corresponding family of second sample. In some embodiments, the quantitative measure of the set of shared families in the first sample excludes shared families at over-represented pairs of genomic start positions and genomic stop positions. In some embodiments, a total number of families in the first sample excludes the families at over-represented pairs of genomic start positions and genomic stop positions. In some embodiments, the over-represented pairs of genomic start positions and genomic stop positions are determined by: (a) providing a plurality of samples, wherein the plurality of samples comprises a distribution of genomic start positions and genomic stop positions that are identical or substantially identical to the first sample and/or the second sample; (b) determining the families in the plurality of samples; (c) quantifying number of families in the plurality of samples sharing a pair of genomic start position and genomic stop position; and (d) categorizing the pair of genomic start position and genomic stop position as over-represented if the number of families exceeds a set threshold. In some embodiments, wherein the plurality of samples excludes the first sample or the second sample. In some embodiments, the plurality of samples excludes the first sample and the second sample. In some embodiments, the plurality of samples comprises samples processed in the same flow cell as the first sample. In some embodiments, the plurality of samples comprises training samples. In some embodiments, the set threshold is at least 2, at least 3, at least 4, at least 5, at least 6, at least 7, at least 8, at least 10, at least 15, at least 20, at least 25, at least 30, at least 35, at least 40, at least 45, at least 50, at least 55 or at least 60 families. In some embodiments, the set threshold is about 5 families. In some embodiments, the set threshold is about 10 families. In some embodiments, the set threshold is about 15 families. In some embodiments, the set threshold is about 20 families. In some embodiments, the set threshold is about 30 families. In some embodiments, the set threshold is about 40 families. In some embodiments, the set threshold is about 50 families. In some embodiments, the set threshold can be at least 10⁻³, at least 10⁻⁴, at least 10⁻⁵, at least 10⁻⁶, at least 10⁻⁷, at least 10⁻⁸, or at least 10⁻⁹ of total families observed in the plurality of samples. In some embodiments, the set threshold can be about 10⁻⁴ of total families observed in the plurality of samples. In some embodiments, the set threshold can be about 10⁻⁵ of total families observed in the plurality of samples. In some embodiments, the set threshold can be about 10⁻⁶ of total families observed in the plurality of samples. In some embodiments, the set threshold can be about 10⁻⁷ of total families observed in the plurality of samples. In some embodiments, the set threshold can be about 10⁻⁸ of total families observed in the plurality of samples.

In 206, the first sample is classified as being contaminated with the second sample, if the quantitative measure of the shared family identifiers is above a predetermined threshold or not contaminated if the quantitative measure of shared family identifiers is at or below the predetermined threshold. In some embodiments, the predetermined threshold is at least 0.001%, at least 0.005%, at least 0.01%, at least 0.05%, at least 0.1%, at least 0.5%, at least 1%, at least 2%, at least 5%, or at least 10% of total number of families in the first sample. In some embodiments, the predetermined threshold is about 0.01% of total number of families in the first sample. In some embodiments, the predetermined threshold is about 0.05% of total number of families in the first sample. In some embodiments, the predetermined threshold is about 0.1% of total number of families in the first sample. In some embodiments, the predetermined threshold is about 0.5% of total number of families in the first sample. In some embodiments, the predetermined threshold is about 1% of total number of families in the first sample. In some embodiments, the predetermined threshold is about 2% of total number of families in the first sample.

In some embodiments, even if the first sample is classified as being contaminated with the second sample, the method can further detect at least one somatic genetic variation of the polynucleotides of the first sample by excluding the sequencing reads of the shared families of the first sample, wherein the first sample is classified as being contaminated with the second sample.

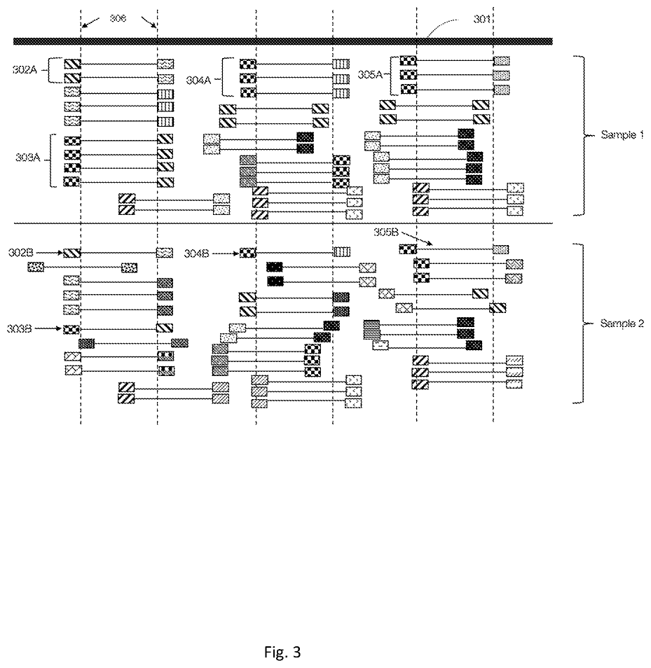

FIG. 3 is a schematic diagram illustrating the grouping of sequencing reads into families and thereby detecting the presence or absence of contamination between two samples (Sample 1 and Sample 2) according to an embodiment of the disclosure. 301 represents the reference sequence (e.g., hG18 or hG19) to which the sequencing reads of Sample 1 and Sample 2 are aligned. For easy illustration, the read1 and read2 of the sequencing reads generated by paired end sequencing from a sequencer is shown as a single paired-end sequencing read, where the read 1 and read 2 sequence reads are merged together. The lines with pattern-filled boxes on both the ends of the line represents paired-end sequencing read (read1+read2). The boxes filled with patterns represent molecular barcodes, which have been attached to both ends of the polynucleotides. Each different pattern represents a different molecular barcode sequence. The paired-end sequencing reads are grouped into families based on the grouping features. In this embodiment, the grouping features are (i) the tag (i.e. molecular barcode); (ii) the start position and (iii) the stop position of the polynucleotide.

302A, 303A, 304A and 305A are shared families of Sample 1 as the grouping features of those families are identical or substantially identical to the grouping features of families 302B, 303B, 304B and 305B respectively of Sample 2. Similarly, 302B, 303B, 304B and 305B are shared families of Sample 2 as the grouping features of those families are identical or substantially identical to the grouping features of families 302A, 303A, 304A and 305A respectively of Sample 1. 306 represents a pair of genomic start and stop positions. At 306, Sample 1 has three families and Sample 2 has four families, and hence the total number of families at 306 is seven. In this embodiment, to determine if a particular pair of genomic start and genomic stop positions is an over-represented pair, the set threshold value is six. Since the total number of families (i.e., seven) at 306 is above the set threshold, 306 is an over-represented pair of genomic start and stop position.

Scenario I:

Determining if Sample 1 is contaminated with Sample 2.

The number of shared families in Sample 1 is four (302A, 303A, 304A and 305A), out of which two families 302A and 303A are in the over-represented pair of genomic start and genomic stop positions. In this embodiment, for determining the quantitative measure of the shared families in sample 1, the shared families of Sample 1 at the over-represented pairs of genomic start positions and genomic stop positions are excluded. Since 306 is an over-represented pair, two families (302A and 303A) are excluded in calculating the quantitative measure of the shared families. Therefore, the quantitative measure of shared families for Sample 1 is two. In this embodiment, the quantitative measure also excludes the shared families in Sample 1 for which the number of sequencing reads in the family of sample 1 is greater than the number of sequencing reads in the corresponding family of Sample 2. In this embodiment, shared families of sample 1 (304A and 305A) have three paired-end sequencing reads (i.e., six sequencing reads) each, whereas the corresponding families of sample 2 (304B and 305B) have one paired-end sequencing read (i.e., two sequencing reads) each. Accordingly, shared families 304A and 305A are excluded from calculating the quantitative measure. Hence, the quantitative measure of the shared families in Sample 1 is zero. To classify Sample 1 as being contaminated with Sample 2, the quantitative measure of the shared families should be above the predetermined threshold. In this embodiment, the predetermined threshold is 0.5% of total families. Since the quantitative measure (i.e. zero for the first sample) is below the predetermined threshold, Sample 1 is determined not to be contaminated with Sample 2.

Scenario II:

Determining if Sample 2 is contaminated with Sample 1

The number of shared families in Sample 2 is four (302B, 303B, 304B and 305B), out of which two families 302B and 303B are in the over-represented pair of genomic start and genomic stop positions. In this embodiment, for determining the quantitative measure of the shared families in Sample 2, the shared families of Sample 2 at the over-represented pairs of genomic start and genomic stop positions are excluded. Since 306 is an over-represented pair, two families (302B and 303B) are excluded in determining the quantitative measure of the shared families. Therefore, the quantitative measure of shared families for Sample 2 is two. In this embodiment, the quantitative measure also excludes the shared families of Sample 2 for which the number of sequencing reads in the family of Sample 2 is greater than the number of sequencing reads in the corresponding family of Sample 1. In this embodiment, shared families of Sample 2 (304B and 305B) have one paired-end sequencing read (i.e., two sequencing reads) each, whereas the corresponding families in Sample 1 (304A and 305A) have three paired-end sequencing reads (i.e., six sequencing reads) each. Accordingly, shared families 304A and 305A are not excluded from calculating the quantitative measure. Hence, the quantitative measure of the shared families in Sample 2 is two. To classify Sample 2 as being contaminated with Sample 1, the quantitative measure of the shared families of Sample 2 should be above the predetermined threshold. In this embodiment, the predetermined threshold is 0.5% of total families. For Sample 2, the total number of families is 21. In this embodiment, the families at the over-represented pairs of genomic start position and genomic start positions are excluded form the total number of families. The number of families at over-represented pair of genomic start and genomic stop positions 306 is 4. So the total number of families in Sample 2 after excluding the families at over-represented pair is 17. Also, in this embodiment, the quantitative measure of the shared families is the percentage of the total families in Sample 2 which were shared families, which is equal to 11.765% (100*2/17) and it is above the predetermined threshold. Therefore, Sample 2 is determined to be contaminated with Sample 1.

The various steps of the methods may be carried out the same or different times, in the same or different geographical locations, e.g. countries, and by the same or different people or entities.

### II. General Features of the Methods

A. Samples

A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA). In some embodiments, the methods include obtaining the sample from a subject. Essentially any sample type is optionally utilized. In certain embodiments, for example, the sample is tissue, blood, plasma, serum, sputum, urine, semen, vaginal fluid, feces, synovial fluid, spinal fluid, saliva, and/or the like. Typically, the subject is a mammalian subject (e.g., a human subject). In some embodiments, the sample is blood. In some embodiments, the sample is plasma. In some embodiments, the sample is serum.

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equates with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some embodiments, the sample comprises cell-free DNA (i.e., cfDNA sample). In some embodiments, the cfDNA sample comprises circulating tumor nucleic acids.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples. In certain embodiments, methods include obtaining between about 5 ng to about 30 ng of cell-free nucleic acid molecules from samples. In certain embodiments, methods include obtaining between about 5 ng to about 100 ng of cell-free nucleic acid molecules from samples. In certain embodiments, methods include obtaining between about 5 ng to about 150 ng of cell-free nucleic acid molecules from samples. In certain embodiments, methods include obtaining between about 5 ng to about 200 ng of cell-free nucleic acid molecules from samples. In some embodiments, the amount is up to about 100 ng of cell-free nucleic acid molecules from samples. In some embodiments, the amount is up to about 150 ng of cell-free nucleic acid molecules from samples. In some embodiments, the amount is up to about 200 ng of cell-free nucleic acid molecules from samples. In some embodiments, the amount is up to about 250 ng of cell-free nucleic acid molecules from samples. In some embodiments, the amount is up to about 300 ng of cell-free nucleic acid molecules from samples. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides in length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps.

B. Nucleic Acid Tags

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) portions corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcodes, or from about 5 to about 150 different molecular barcodes, or from about 20 to about 50 different molecular barcodes, ligated to both ends of a target molecule. Alternatively, from about 25 to about 1,000,000 different molecular barcodes may be used. For example, 20-50×20-50 molecular barcodes can be used. In some embodiments, 20-50 different molecular barcodes can be used. In some embodiments, 5-100 different molecular barcodes can be used, In some embodiments, 5-150 molecular barcodes can be used. In some embodiments, 5-200 different molecular barcodes can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

C. Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other amplification exemplary methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular barcodes and/or sample indexes to a nucleic acid molecule using conventional nucleic acid amplification methods. The amplifications are typically conducted in one or more reaction mixtures. Molecular barcodes and sample indexes are optionally introduced simultaneously, or in any sequential order. In some embodiments, molecular barcodes and sample indexes are introduced prior to and/or after sequence capturing steps are performed. In some embodiments, only the molecular barcodes are introduced prior to probe capturing and the sample indexes are introduced after sequence capturing steps are performed. In certain embodiments, both the molecular barcodes and the sample indexes are introduced prior to performing probe-based capturing steps. In some embodiments, the sample indexes are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region is associated with a cancer type. Typically, the amplification reactions generate a plurality of non-uniquely or uniquely tagged nucleic acid amplicons with molecular barcodes and sample indexes at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

D. Enrichment

Sequences can be enriched prior to sequencing. Enrichment can be performed for specific target regions or nonspecifically (“target sequences”). In some embodiments, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include natural or synthetic nucleotide sequences of the nucleic acid construct. In some embodiments, biotin-labeled beads with probes to one or more regions of interest can be used to capture target sequences, optionally followed by amplification of those regions, to enrich for the regions of interest.

Sequence capture may comprise the use of oligonucleotide probes that hybridize to the target sequence. A probe set strategy can involve tiling the probes across a region of interest. Such probes can be, e.g., about 60 to 120 bases long. The set can have a depth of about 2×, 3×, 4×, 5×, 6×, 8×, 9×, 10×, 15×, 20×, 50×, or more than 50×. The effectiveness of sequence capture depends, in part, on the length of the sequence in the target molecule that is complementary (or nearly complementary) to the sequence of the probe.

In some embodiments, the plurality of genomic regions comprises genetic variants found in COSMIC, The Cancer Genome Atlas (TCGA), or the Exome Aggregation Consortium (ExAC). In some cases, genetic variants may belong to a pre-defined set of clinically actionable variants. For example, such variants may be found in various databases of variants whose presence in a sample of a subject have been shown to correlate with or be indicative of a disease or disorder (e.g., cancer) in the subject. Such databases of variants may include, for example, the Catalogue of Somatic Mutations in Cancer (COSMIC), The Cancer Genome Atlas (TCGA), and the Exome Aggregation Consortium (ExAC). A pre-defined set of such catalogued variants may be designated for further bioinformatics analysis due to their relevance to clinical decision-making (e.g., diagnosis, prognosis, treatment selection, targeted treatment, treatment monitoring, monitoring for recurrence, etc.). Such a pre-defined set may be determined based on, for example, analysis of clinical samples (e.g., of patient cohorts with known presence or absence of a disease or disorder) as well as annotation information from public databases and clinical literature.

E. Sequencing

Sample nucleic acids flanked by adapters with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

The sequencing reactions can be performed on one or more nucleic acid fragment types or regions known to contain markers of cancer or other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9% or 100% of the genome. In other cases, sequence reactions may be performed on less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some cases, cell free polynucleotides may be sequenced with at least 1000, 2000, 3000, 4000, 5000, 6000, 7000, 8000, 9000, 10000, 50000, or 100,000 sequencing reactions. In other cases, cell free polynucleotides may be sequenced with less than 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, or 100,000 sequencing reactions. In other cases, data analysis may be performed on less than 1000, 2000, 3000, 4000, 5000, 6000, 7000, 8000, 9000, 10000, 50000, or 100,000 sequencing reactions. An exemplary read depth is 1000-50000 reads per locus (base). In some embodiments, read depth can be greater than 50000 reads per locus (base).

F. Analysis

Sequencing according to embodiments of the invention generates a plurality of sequencing reads or reads. Sequencing reads or reads according to the invention generally include sequences of nucleotide data less than about 150 bases in length, or less than about 90 bases in length. In certain embodiments, reads are between about 80 and about 90 bases, e.g., about 85 bases in length. In some embodiments, methods of the invention are applied to very short reads, i.e., less than about 50 or about 30 bases in length. Sequencing read data can include the sequence data as well as meta information. Sequence read data can be stored in any suitable file format including, for example, VCF files, FASTA files or FASTQ files.

FASTA is originally a computer program for searching sequence databases and the name FASTA has come to also refer to a standard file format. See Pearson & Lipman, 1988, Improved tools for biological sequence comparison, PNAS 85:2444-2448. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer, as described by, for example, Cock et al. (“The Sanger FASTQ file format for sequences with quality scores, and the Solexa/Illumina FASTQ variants,” Nucleic Acids Res 38(6):1767-1771, 2009), which is hereby incorporated by reference in its entirety.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “-”. In a preferred embodiment, the sequence data will use the A, T, C, G, and N characters, optionally including “-” or U as-needed (e.g., to represent gaps or uracil).

In some embodiments, the at least one master sequence read file and the output file are stored as plain text files (e.g., using encoding such as ASCII; ISO/IEC 646; EBCDIC; UTF-8; or UTF-16). A computer system provided by the invention may include a text editor program capable of opening the plain text files. A text editor program may refer to a computer program capable of presenting contents of a text file (such as a plain text file) on a computer screen, allowing a human to edit the text (e.g., using a monitor, keyboard, and mouse). Exemplary text editors include, without limit, Microsoft Word, emacs, pico, vi, BBEdit, and TextWrangler. Preferably, the text editor program is capable of displaying the plain text files on a computer screen, showing the meta information and the sequence reads in a human-readable format (e.g., not binary encoded but instead using alphanumeric characters as they may be used in print human writing).

While methods have been discussed with reference to FASTA or FASTQ files, methods and systems of the invention may be used to compress any suitable sequence file format including, for example, files in the Variant Call Format (VCF) format. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by Danecek et al. (“The variant call format and VCFtools,” Bioinformatics 27(15):2156-2158, 2011), which is hereby incorporated by reference in its entirety. The header section may be treated as the meta information to write to the compressed files and the data section may be treated as the lines, each of which will be stored in a master file only if unique.

Certain embodiments of the invention provide for the assembly of sequencing reads. In assembly by alignment, for example, the sequencing reads are aligned to each other or aligned to a reference sequence. By aligning each read, in turn to a reference genome, all of the reads are positioned in relationship to each other to create the assembly. In addition, aligning or mapping the sequencing read to a reference sequence can also be used to identify variant sequences within the sequencing read. Identifying variant sequences can be used in combination with the methods and systems described herein to further aid in the diagnosis or prognosis of a disease or condition, or for guiding treatment decisions.

In some embodiments, any or all of the steps are automated. Alternatively, methods of the invention may be embodied wholly or partially in one or more dedicated programs, for example, each optionally written in a compiled language such as C++ then compiled and distributed as a binary. Methods of the invention may be implemented wholly or in part as modules within, or by invoking functionality within, existing sequence analysis platforms. In certain embodiments, methods of the invention include a number of steps that are all invoked automatically responsive to a single starting cue (e.g., one or a combination of triggering events sourced from human activity, another computer program, or a machine). Thus, the invention provides methods in which any or the steps or any combination of the steps can occur automatically responsive to a cue. Automatically generally means without intervening human input, influence, or interaction (i.e., responsive only to original or pre-cue human activity).

The system also encompasses various forms of output, which includes an accurate and sensitive interpretation of the subject nucleic acid. The output of retrieval can be provided in the format of a computer file. In certain embodiments, the output is a FASTA file, FASTQ file, or VCF file. Output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact Idiosyncratic Gapped Alignment Report (CIGAR) (Ning et al., Genome Research 11(10):1725-9, 2001, which is hereby incorporated by reference in its entirety). These strings are implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file—comprising a CIGAR string (the SAM format is described, e.g., by Li et al., “The Sequence Alignment/Map format and SAMtools,” Bioinformatics, 25(16):2078-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save some space), 3 matches, 2 deletions and 2 matches.

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U) in the form of dNTPs. Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including molecular barcodes, and the sequencing determines nucleic acid sequences as well as molecular barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (for e.g., sticky end ligation).

The nucleic acid sample is typically contacted with a sufficient number of adapters that there is a low probability (e.g., <1 or <0.1%) that any two copies of the same nucleic acid receive the same combination of adapter barcodes (i.e., molecular barcodes) from the adapters linked at both ends. The use of adapters in this manner permits identification of families of nucleic acid sequences with the same start and stop points on a reference nucleic acid and linked to the same combination of molecular barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### III. Computer Systems

Methods of the present disclosure can be implemented using, or with the aid of, computer systems. For example, such methods may comprise (a) obtaining a plurality of sequencing reads of the set of tagged polynucleotides from first sample and second sample generated by the nucleic acid sequencer, wherein the sequencing read comprises a tag sequence and a sequence derived from a polynucleotide; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the tag, (ii) the beginning region, (iii) the end region and (iv) length of the polynucleotide, wherein each family in the sample comprises sequencing reads of tagged progeny polynucleotides amplified from a unique polynucleotide among the set of polynucleotides in the sample; (d) generating family identifiers for the plurality of families; (e) screening for a set of shared family identifiers wherein the shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the shared family identifiers is above a predetermined threshold, or not contaminated if the quantitative measure of shared family identifiers is at or below the predetermined threshold, can be performed with a computer processor.

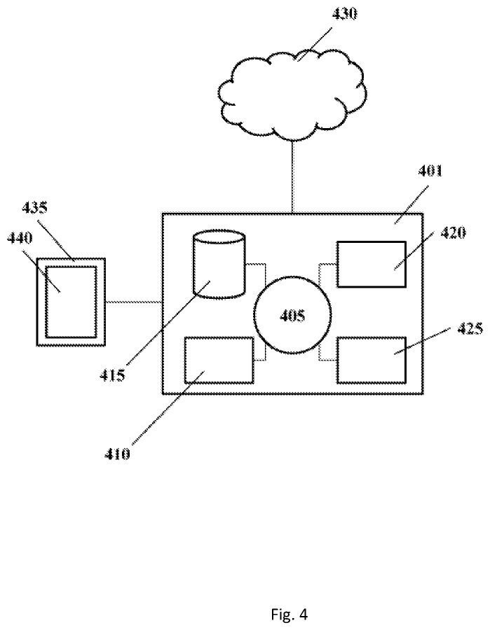

FIG. 4 shows a computer system 401 that is programmed or otherwise configured to implement the methods of the present disclosure. The computer system 401 can regulate various aspects sample preparation, sequencing, and/or analysis. In some examples, the computer system 401 is configured to perform sample preparation and sample analysis, including nucleic acid sequencing.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage, and/or electronic display adapters. The memory 410, storage unit 415, interface 420, and peripheral devices 425 are in communication with the CPU 405 through a communication network or bus (solid lines), such as a motherboard. The storage unit 415 can be a data storage unit (or data repository) for storing data. The computer system 401 can be operatively coupled to a computer network 430 with the aid of the communication interface 420. The computer network 430 can be the Internet, an internet and/or extranet, or an intranet and/or extranet that is in communication with the Internet. The computer network 430 in some cases is a telecommunication and/or data network. The computer network 430 can include one or more computer servers, which can enable distributed computing, such as cloud computing. The computer network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. Examples of operations performed by the CPU 405 can include fetch, decode, execute, and writeback.

The storage unit 415 can store files, such as drivers, libraries, and saved programs. The storage unit 415 can store programs generated by users and recorded sessions, as well as output(s) associated with the programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet. Data may be transferred from one location to another using, for example, a communication network or physical data transfer (e.g., using a hard drive, thumb drive, or other data storage mechanism).

The computer system 401 can communicate with one or more remote computer systems through the network 430. For instance, the computer system 401 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

In an aspect, the present disclosure provides a non-transitory computer-readable medium comprising computer-executable instructions which, when executed by at least one electronic processor, perform a method comprising: (a) obtaining a plurality of sequencing reads of the set of tagged polynucleotides from first sample and second sample generated by the nucleic acid sequencer, wherein the sequencing read comprises a tag sequence and a sequence derived from a polynucleotide; (b) aligning the plurality of sequencing reads to a reference sequence whereby a beginning region and an end region of the alignment is determined; (c) for each sample, grouping the plurality of sequencing reads into a plurality of families based on grouping features, which comprise at least one of (i) the tag, (ii) the beginning region, (iii) the end region and (iv) length of the polynucleotide, wherein each family in the sample comprises sequencing reads of tagged progeny polynucleotides amplified from a unique polynucleotide among the set of polynucleotides in the sample; (d) generating family identifiers for the plurality of families; (e) screening for a set of shared family identifiers wherein the shared family identifier is a family identifier of the first sample that is identical or substantially identical to a family identifier of the second sample; (f) determining a quantitative measure of the set of shared family identifiers; and (g) classifying the first sample as being contaminated with the second sample if the quantitative measure of the shared family identifiers is above a predetermined threshold, or not contaminated if the quantitative measure of shared family identifiers is at or below the predetermined threshold.

The code can be pre-compiled and configured for use with a machine have a processor adapted to execute the code or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

Applications

Cancer and Other Diseases

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Although the description has been described with respect to particular embodiments thereof, these particular embodiments are merely illustrative, and not restrictive. Concepts illustrated in the examples may be applied to other examples and implementations.

As liquid biopsy assays are changed (e.g., in sequencing depth and panels of common SNPs), methods and systems of the present disclosure may be retrained as needed to obtain a set of applicable threshold values (for example, one or more criteria/threshold to detect the presence or absence of a contamination in a sample).

### EXAMPLES

### Example 1: To Determine the Contamination of Samples According to an Embodiment of the Disclosure

A set of patient samples were analyzed using a blood-based cfDNA assay at Guardant Health (Redwood City, Calif., USA). To check the quality of the assay performance and to determine if there is any contamination of samples, the set of samples were analyzed according to an embodiment of the disclosure. Among the set of samples, the analysis of two samples (Sample 1 and Sample 2) is described in this example. The total number of families in Sample 1 and Sample 2 are U.S. Pat. Nos. 7,811,148 and 7,141,008 respectively. In this embodiment, families at the over-represented pairs of genomic start and genomic stop positions were excluded from the analysis and the set threshold used to categorize a pair of genomic start position and genomic stop position as over-represented pair is 10 families. So, the total number of families in Sample 1 and Sample 2 were 6,452,057 and 6,039,099 respectively.

I: To Determine if Sample 1 was Contaminated with Sample 2

Out of 6,452,057 families in Sample 1, 54,212 families were shared families (with Sample 2). Out of the 54,212 shared families: (i) 9362 shared families had the same number of sequencing reads within the families in both Sample 1 and Sample 2; and (ii) 1647 shared families had a greater number of sequencing reads in the families of Sample 1 than the number of sequencing reads in the corresponding families of Sample 2. In this embodiment, the shared families with a greater number of sequencing reads in the families of Sample 1 than the number of sequencing reads in the corresponding families of Sample 2 were excluded in determining the quantitative measure of the shared families. Also, in this embodiment, the quantitative measure of the shared families was the percentage of the total families in Sample 1 which were shared families, which was equal to 0.815% (100*(54212−1647)/6452057). In this embodiment, the predetermined threshold to classify a sample as being contaminated was 0.5%. Since the quantitative measure of the shared families of Sample 1 was greater than 0.5%, Sample 1 was determined to be contaminated with Sample 2.

II: To Determine if Sample 2 was Contaminated with Sample 1

Out of 6,039,099 families in Sample 2, 54,212 families were shared families (with Sample 1). Out of the 54,212 shared families: (i) 9362 shared families had the same number of sequencing reads within the family in both Sample 1 and Sample 2; and (ii) 43,203 shared families had a greater number of sequencing reads in the families of Sample 2 than the number of sequencing reads in the corresponding families of Sample 1. Excluding the shared families with a greater number of sequencing reads in the families of Sample 2 than the number of sequencing reads in the corresponding families Sample 1, the quantitative measure of the shared families of Sample 2 was equal to 0.182% (100*(54212−43203)/6039099). Since the quantitative measure of the shared families of Sample 2 was lower than the predetermined threshold (0.5%), Sample 2 was determined to be not contaminated with Sample 1.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

1.-62. (canceled)

63. A method for detecting the presence or absence of contamination of a first sample with a second sample among a plurality of samples, comprising:
(a) processing the first sample and the second sample, wherein the processing comprises:
i. tagging a set of cell-free nucleic acid molecules in each sample with a set of molecular barcodes to generate tagged polynucleotides, wherein the set of molecular barcodes comprise 5-200 different molecular barcode sequences;
ii. amplifying a portion of the tagged polynucleotides to generate progeny polynucleotides;
(b) for each of the first sample and the second sample, sequencing a portion of the progeny polynucleotides to generate sequencing reads;
(c) aligning a plurality of sequencing reads from the first sample and the second sample to a reference sequence whereby a genomic start position and a genomic stop position of the cell-free nucleic acid molecule is determined from the alignment;
(d) for each of the first sample and the second sample, grouping the plurality of sequencing reads into a plurality of families based on grouping features, which comprise at least one of (i) one or more molecular barcodes attached to a cell-free nucleic acid molecule in the sample, (ii) start genomic position and (iii) stop genomic position of the cell-free nucleic acid molecule, wherein each family in the sample comprises sequencing reads of progeny polynucleotides amplified from a unique cell-free nucleic acid molecule among the set of cell-free nucleic acid molecules in the sample;
(e) generating family identifiers for the plurality of families;
(f) screening for a set of shared family identifiers, wherein a given shared family identifier is a family identifier of the first sample that is identical or substantially identical to a family identifier of the second sample;
(g) determining a quantitative measure of the set of shared family identifiers; and
(h) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold, thereby detecting the presence or absence of contamination.

64. The method of claim 63, wherein the quantitative measure of the set of shared family identifiers is a number of shared family identifiers in the first sample.

65. The method of claim 63, wherein the quantitative measure of the set of shared family identifiers excludes shared family identifiers in the first sample for which the number of sequencing reads in the family of the first sample is greater than the number of sequencing reads in the corresponding family of the second family.

66. The method of claim 63, wherein the quantitative measure of the set of shared family identifiers in the first sample excludes shared family identifiers at over-represented pairs of genomic start positions and genomic stop positions.

67. The method of claim 66, wherein the over-represented pairs of genomic start positions and genomic stop positions are determined by:
(a) providing sets of sequencing reads from the plurality of samples, wherein the sets of sequencing reads comprise a distribution of genomic start positions and genomic stop positions that are identical or substantially identical to the first sample;
(b) determining family identifiers in the sets of sequencing reads;
(c) quantifying number of family identifiers in the sets of sequencing reads sharing a pair of genomic start position and genomic stop position; and
(d) categorizing the pair of genomic start position and genomic stop position as over-represented if the number of family identifiers exceeds a set threshold.

68. The method of claim 63, wherein the plurality of samples comprises samples processed in a same flow cell as the first sample.

69. The method of claim 67, wherein the set threshold is at least 5, at least 10, at least 15 or at least 20.

70. The method of claim 63, wherein the one or more molecular barcodes are attached to both ends of the cell-free nucleic acid molecule.

71. The method of claim 63, wherein the first sample and the second sample are sequenced in a same flow cell.

72. The method of claim 63, wherein the processing, further comprises, enriching a portion of the progeny polynucleotides for specific regions of interest to generate enriched molecules.

73. The method of claim 63, wherein one or more sample indexes are attached to one or both ends of the progeny polynucleotides prior to the sequencing, wherein the one or more sample indexes distinguishes the first sample and the second sample.

74. The method of claim 63, wherein the predetermined threshold is at least 0.5% or at least 1% of total number of families in the first sample.

75. The method of claim 63, wherein the first sample is obtained from a bodily fluid of a subject and the second sample is obtained from the bodily fluid of another subject.

76. The method of claim 75, wherein the bodily fluid is plasma.

77. A computer-implemented method for detecting the presence or absence of contamination of a first sample with a second sample among a plurality of samples, comprising:
(a) obtaining sequence information comprising a plurality of sequencing reads derived from a set of cell-free nucleic acid molecules from the first sample and another set of cell-free nucleic acid molecules from the second sample;
(b) aligning the plurality of sequencing reads to a reference sequence whereby a genomic start position and a genomic stop position of the cell-free nucleic acid molecule is determined from the alignment;
(c) for each of the first sample and the second sample, grouping the plurality of sequencing reads into a plurality of families based on grouping features, which comprise at least one of (i) one or more molecular barcodes attached to a cell-free nucleic acid molecule in the sample, (ii) start genomic position and (iii) stop genomic position of the cell-free nucleic acid molecule, wherein each family in the sample comprises sequencing reads of progeny polynucleotides amplified from a unique cell-free nucleic acid molecule among the set of cell-free nucleic acid molecules in the sample;
(d) generating family identifiers for the plurality of families;
(e) screening for a set of shared family identifiers, wherein a given shared family identifier is a family identifier of the first sample that is identical or substantially identical to a family identifier of the second sample;
(f) determining a quantitative measure of the set of shared family identifiers; and
(g) classifying the first sample as being contaminated with the second sample if the quantitative measure of the set of shared family identifiers is above a predetermined threshold, or as not being contaminated with the second sample if the quantitative measure of the set of shared family identifiers is at or below the predetermined threshold, thereby detecting the presence or absence of contamination.

78. The method of claim 77, wherein the quantitative measure of the set of shared family identifiers is a number of shared family identifiers in the first sample.

79. The method of claim 77, wherein the quantitative measure of the set of shared family identifiers excludes shared family identifiers in the first sample for which the number of sequencing reads in the family of the first sample is greater than the number of sequencing reads in the corresponding family of the second family.

80. The method of claim 77, wherein the quantitative measure of the set of shared family identifiers in the first sample excludes shared family identifiers at over-represented pairs of genomic start positions and genomic stop positions.

81. The method of claim 80, wherein the over-represented pairs of genomic start positions and genomic stop positions are determined by:
(a) providing sets of sequencing reads from the plurality of samples, wherein the sets of sequencing reads comprise a distribution of genomic start positions and genomic stop positions that are identical or substantially identical to the first sample;
(b) determining family identifiers in the sets of sequencing reads;
(c) quantifying number of family identifiers in the sets of sequencing reads sharing a pair of genomic start position and genomic stop position; and
(d) categorizing the pair of genomic start position and genomic stop position as over-represented if the number of family identifiers exceeds a set threshold.

82. The method of claim 81, wherein the plurality of samples comprises sample processed in a same flow cell as the first sample.
